(19) United States
(12) Patent Application Publication
(10) Pub. No.: US 2016/0194716 A1
(43) Pub. Date: Jul. 7, 2016

(54) MIRNA in Gulf War Illness

(71) Applicant: GEORGETOWN UNIVERSITY, Washington, DC (US)

(72) Inventors: James N. Baraniuk, Bethesda, MD (US); Narayan Shivapurkar, Potomac Falls, VA (US); Rakib Rayhan, Washington, DC (US)

(73) Assignee: GEORGETOWN UNIVERSITY, Washington, DC (US)

(21) Appl. No.: 14/983,817

(22) Filed: Dec. 30, 2015

Related U.S. Application Data
(60) Provisional application No. 62/099,767, filed on Jan. 5, 2015.

(51) Int. Cl. C12Q 1/68

(52) U.S. Cl. CPC C12Q 1/6883; C12Q 2600/158; C12Q 2600/178; C12Q 2600/16

(57) ABSTRACT

Provided herein is a method of determining a level of one or more miRNAs in a subject that has or is at risk of developing Gulf War Illness (GWI). The method includes obtaining a biological sample from the subject and determining a level of one or more miRNA molecules in the biological sample. Also provided are compositions and kits for carrying out the provided methods.

### CROSS-REFERENCE TO RELATED APPLICATION

This application claims priority to U.S. Provisional Application No. 62/099,767, filed Jan. 5, 2015, which is incorporated herein by reference in its entirety.

### SEQUENCE LISTING

In accordance with 37 CFR §1.52(e)(5), the present specification makes reference to a Sequence Listing (entitled “GEO_051US1_0968254_ST25.txt”, created on Dec. 29, 2015, and 8.44 KB). The entire contents of the Sequence Listing are herein incorporated by reference.

### BACKGROUND

The diagnostic criteria for Gulf War Illness (GWI) are controversial. GWI subjects can be divided into subsets based on criteria such as exercise-induced changes in postural tachycardia, brain stem atrophy, brain blood flow, brain lactate levels, and exercise-induced changes in accuracy on cognitive testing. Specifically, GWI can be divided into at least two subsets, START (Stress Test Activated Reversible Tachycardia) subjects and STOPP (Stress Test Originated Phantom Perception) subjects.

### BRIEF SUMMARY

Provided herein is a method of determining a level of one or more miRNAs in a subject that has or is at risk of developing Gulf War Illness (GWI). The method includes obtaining a biological sample from the subject and determining a level of one or more miRNA molecules in the biological sample. Also provided are compositions and kits for carrying out the provided methods.

The details of one or more embodiments are set forth in the accompanying drawings and the description below. Other features, objects, and advantages will be apparent from the description and drawings, and from the claims.

### BRIEF DESCRIPTION OF THE DRAWING

FIG. 1 is a schematic showing the 3 dimension model of objective GWI outcomes.

### DETAILED DESCRIPTION

Gulf War Illness (GWI) is a chronic multisymptom disorder affecting returning military veterans and civilian workers of the Gulf War. A wide range of acute and chronic symptoms have been associated with GWI, including fatigue, muscle pain, cognitive problems, rashes and diarrhea.

Provided herein is a method of determining a level of one or more miRNAs in a subject that has or is at risk of developing Gulf War Illness (GWI). The method includes obtaining a biological sample from the subject and determining a level of one or more miRNAs in the biological sample. Optionally, the miRNA is hsa-let-7a-5p (SEQ ID NO:1), hsa-let-7c-5p (SEQ ID NO:2), hsa-let-7d-5p (SEQ ID NO:3), hsa-let-7d-3p (SEQ ID NO:4), hsa-let-7e-5p (SEQ ID NO:5), hsa-miR-10a-5p (SEQ ID NO:6), hsa-miR-10a-3p (SEQ ID NO:7), hsa-miR-10b-5p (SEQ ID NO:8), hsa-miR-10b-3p (SEQ ID NO:9), hsa-miR-129-2-3p (SEQ ID NO:10), hsa-miR-129-5p (SEQ ID NO:11), hsa-miR-130a-3p (SEQ ID NO:12), hsa-miR-136-5p (SEQ ID NO:13), hsa-miR-141-3p (SEQ ID NO:14), hsa-miR-142-3p (SEQ ID NO:15), hsa-miR-142-5p (SEQ ID NO:16), hsa-miR-155-5p (SEQ ID NO:17), hsa-miR-155-3p (SEQ ID NO:18), hsa-miR-17-5p (SEQ ID NO:19), hsa-miR-182-5p (SEQ ID NO:20), hsa-miR-182-3p (SEQ ID NO:21), hsa-miR-183-5p (SEQ ID NO:22), hsa-miR-186-5p (SEQ ID NO:23), hsa-miR-186-3p (SEQ ID NO:24), hsa-miR-200a-3p (SEQ ID NO:(25), hsa-miR-200a-5p (SEQ ID NO:26), hsa-miR-200 b-3p (SEQ ID NO:27), hsa-miR-200c-3p (SEQ ID NO:28), hsa-miR-200c-5p (SEQ ID NO:29), hsa-miR-204-5p (SEQ ID NO:30), hsa-miR-20a-5p (SEQ ID NO:31), hsa-miR-20a-3p (SEQ ID NO:32), hsa-miR-22-3p (SEQ ID NO:33), hsa-miR-223-3p (SEQ ID NO:34), hsa-miR-223-5p (SEQ ID NO:35), hsa-miR-27a-3p (SEQ ID NO:36), hsa-miR-27a-5p (SEQ ID NO:37), hsa-miR-27b-3p (SEQ ID NO:38), hsa-miR-27b-5p (SEQ ID NO:39), hsa-miR-30a-5p (SEQ ID NO:40), hsa-miR-30d-5p (SEQ ID NO:41), hsa-miR-30d-3p (SEQ ID NO:42), hsa-miR-30e-5p (SEQ ID NO:43), hsa-miR-373-3p (SEQ ID NO:44), hsa-miR-373-5p (SEQ ID NO:45), hsa-miR-383-5p (SEQ ID NO:46), hsa-miR-432-5p (SEQ ID NO:47), hsa-miR-483-5p (SEQ ID NO:48), hsa-miR-486-3p (SEQ ID NO:49), hsa-miR-486-5p (SEQ ID NO:50), hsa-miR-92 b-3p (SEQ ID NO:51), hsa-miR-92b-5 (SEQ ID NO:52), hsa-miR-93-5p (SEQ ID NO:53), hsa-miR-93-3p (SEQ ID NO:54), hsa-miR-99b-5p (SEQ ID NO:55), hsa-miR-4763 (SEQ ID NO:56), hsa-miR-1298 (SEQ ID NO:57) or a combination thereof. Optionally, the miRNAs are miRNAs miR-141, miR-200b, miR-223, miR-130a, miR-155, miR-20a, miR-10a, or any combination thereof. Optionally, the level of one or more of miR-141, miR-200b-miR-223, miR-130a, miR-155, miR-20a, or miR-10a is reduced as compared to a control. Optionally, the provided methods further comprise determining a level of miR-10b, miR-99b, miR-22, miR-30a, miR-182, miR-LET7c, miR-30d, miR-27b, miR-27a, miR-17, miR-4763, miR-LET7d, miR-93, miR-183, miR-186, miR-486, and miR-LET7a2, or any combination thereof. Optionally, the level of one or more of miR-10b, miR-99b, miR-22, miR-30a, miR-182, miR-LET7c, miR-30d, miR-27b, miR-27a, miR-17, miR-4763, miR-LET7d, miR-93, miR-183, miR-186, miR-486, or miR-LET7a2 is increased as compared to a control. Optionally, the provided methods further include determining a level of miR-142, miR-204, miR-1298, or any combination thereof. Optionally, the level of one or more of miR-142, miR-204, or miR-1298 is increased as compared to a control. Optionally, the provided methods further include determining a level of miR-92b, miR-483, miR-200a, miR-136, miR-129, miR-383, miR-432, miR-373, miR-200c, or any combination thereof. Optionally, the subject has GWI. Optionally, the subject has GWI, subtype stress test activated reversible tachycardia (START). Optionally, the subject has GWI, subtype stress test originated phantom perception (STOPP).

The term miRNA refers to a microRNA molecule found in eukaryotes that is involved in gene regulation. See, e.g., Carrington et al., Science 301(5631):336-8 (2003), which is hereby incorporated by reference. Names of the relevant miRNAs and their sequences are provided herein. MiRNAs are small non-coding RNA molecules of approximately 20 to 25 nucleotides in length that typically base-pair with the 3′ untranslated regions (UTRs) of protein-encoding messenger RNAs (mRNAs). This binding negatively regulates gene expression of the mRNA by leading to degradation or translation blockade of the mRNA. Through regulation of greater than 60% of all protein-coding genes, miRNAs are involved in a variety of biological pathways including proliferation, differentiation, cell growth, cell death, stress resistance, and metabolism. Deregulation of miRNAs has been linked to diseases and disorders including metabolic disorders and cancer. MiRNAs are also detected in virtually all biofluids including serum and plasma as miRNAs can be secreted through microvesicles (such as exosomes, shedding vesicles, and apoptotic bodies) or in complexes with protein or lipid-based carriers. Accumulating evidence indicates that miRNAs can be transferred to neighboring or distant cells through these secretory forms to modulate cell function. Extracellular miRNAs are therefore emerging as a new group of messengers and effectors in intercellular communication.

As used herein, the term miRNA includes all forms of miRNAs including the pri-, pre- and mature forms of an miRNA, as well as variants, modifications and derivatives thereof. As discussed above, miRNAs are typically generated from large RNA precursors (termed pri-miRNAs), which are then processed in the nucleus into smaller length RNA molecules referred to as pre-miRNAs (usually ˜70 nucleotides). Pre-miRNA molecules fold into stem-loop or hairpin structures and undergo an additional processing step within the cytoplasm where mature miRNAs of approximately 18 to 25 nucleotides in length are excised from the pre-miRNA hairpin. As used herein, the term miRNA includes all forms of these miRNA molecules including, but not limited to, the pri-, pre-, and mature forms of miRNA.

The provided methods can further include steps or methods used in testing for GWI including, but not limited to, molecular spectroscopy, measuring exercise-induced heart rate changes, measuring exercise-induced cerebral blood flow, measuring exercise-induced lactate levels, performing magnetic resonance imaging of the brain, or combinations thereof.

The step of determining the levels of miRNA include detecting miRNA in a biological sample. As used herein, biological samples include, but are not limited to, cells, tissues and bodily fluids. Bodily fluids that used to evaluate the presence or absence of the herein disclosed biomarkers include without limitation blood, urine, serum, tears, lymph, bile, cerebrospinal fluid, interstitial fluid, aqueous or vitreous humor, colostrum, sputum, amniotic fluid, saliva, perspiration, transudate, exudate, and synovial fluid. Optionally, the biological sample is a biological fluid. Optionally, the biological sample is selected from the group consisting of cerebrospinal fluid (CSF), brain cells, urine, peripheral blood white cells, blood, plasma, and serum. Optionally, the biological sample comprises natural killer cells, CD4+ cells, CD8+ cells, IL17+ cells, B lymphocytes or combinations thereof.

MiRNA can be separated from other RNA molecules in a biological sample using methods known in the art. Optionally, miRNA are separated from other RNA molecules using chromatography. Optionally, gel chromatography can be performed using a polyacrylamide gel and tube electrophoresis.

Disclosed herein are biomarkers and methods for identifying and using the biomarkers. By biomarker is meant any assayable characteristics or compositions that are used to identify or monitor a condition (e.g., GWI or lack thereof) or a therapy for said condition in a subject or sample. A biomarker is, for example, hsa-let-7a-5p (SEQ ID NO:1), hsa-let-7c-5p (SEQ ID NO:2), hsa-let-7d-5p (SEQ ID NO:3), hsa-let-7d-3p (SEQ ID NO:4), hsa-let-7e-5p (SEQ ID NO:5), hsa-miR-10a-5p (SEQ ID NO:6), hsa-miR-10a-3p (SEQ ID NO:7), hsa-miR-10b-5p (SEQ ID NO:8), hsa-miR-10b-3p (SEQ ID NO:9), hsa-miR-129-2-3p (SEQ ID NO:10), hsa-miR-129-5p (SEQ ID NO:11), hsa-miR-130a-3p (SEQ ID NO:12), hsa-miR-136-5p (SEQ ID NO:13), hsa-miR-141-3p (SEQ ID NO:14), hsa-miR-142-3p (SEQ ID NO:15), hsa-miR-142-5p (SEQ ID NO:16), hsa-miR-155-5p (SEQ ID NO:17), hsa-miR-155-3p (SEQ ID NO:18), hsa-miR-17-5p (SEQ ID NO:19), hsa-miR-182-5p (SEQ ID NO:20), hsa-miR-182-3p (SEQ ID NO:21), hsa-miR-183-5p (SEQ ID NO:22), hsa-miR-186-5p (SEQ ID NO:23), hsa-miR-186-3p (SEQ ID NO:24), hsa-miR-200a-3p (SEQ ID NO:(25), hsa-miR-200a-5p (SEQ ID NO:26), hsa-miR-200 b-3p (SEQ ID NO:27), hsa-miR-200c-3p (SEQ ID NO:28), hsa-miR-200c-5p (SEQ ID NO:29), hsa-miR-204-5p (SEQ ID NO:30), hsa-miR-20a-5p (SEQ ID NO:31), hsa-miR-20a-3p (SEQ ID NO:32), hsa-miR-22-3p (SEQ ID NO:33), hsa-miR-223-3p (SEQ ID NO:34), hsa-miR-223-5p (SEQ ID NO:35), hsa-miR-27a-3p (SEQ ID NO:36), hsa-miR-27a-5p (SEQ ID NO:37), hsa-miR-27b-3p (SEQ ID NO:38), hsa-miR-27b-5p (SEQ ID NO:39), hsa-miR-30a-5p (SEQ ID NO:40), hsa-miR-30d-5p (SEQ ID NO:41), hsa-miR-30d-3p (SEQ ID NO:42), hsa-miR-30e-5p (SEQ ID NO:43), hsa-miR-373-3p (SEQ ID NO:44), hsa-miR-373-5p (SEQ ID NO:45), hsa-miR-383-5p (SEQ ID NO:46), hsa-miR-432-5p (SEQ ID NO:47), hsa-miR-483-5p (SEQ ID NO:48), hsa-miR-486-3p (SEQ ID NO:49), hsa-miR-486-5p (SEQ ID NO:50), hsa-miR-92 b-3p (SEQ ID NO:51), hsa-miR-92b-5 (SEQ ID NO:52), hsa-miR-93-5p (SEQ ID NO:53), hsa-miR-93-3p (SEQ ID NO:54), hsa-miR-99b-5p (SEQ ID NO:55), hsa-miR-4763 (SEQ ID NO:56), hsa-miR-1298 (SEQ ID NO:57) or a combination thereof whose presence, absence, or relative amount is used to identify a condition or status of a condition in a subject or sample. Biomarkers identified herein are measured to determine levels, expression, activity, or to detect variants.

Methods for detecting and determining levels of miRNA molecules including the miRNA molecules discussed herein are known. Thus, the provided methods include determining the level of miRNAs by performing a Northern Blot, RT-PCR, microarray analysis, or sequencing. Methods for detecting and identifying nucleic acids and proteins and interactions between such molecules involve conventional molecular biology, microbiology, and recombinant DNA techniques within the skill of the art. Such techniques are explained fully in the literature (see, e.g., Green and Sambrook, Molecular Cloning: A Laboratory Manual, Fourth Edition 2012, Cold Spring Harbor Laboratory Press, Cold Spring Harbor, N.Y.; Animal Cell Culture, R. I. Freshney, ed., 1986).

Methods for detecting RNA are largely cumulative with the nucleic acid detection assays and include, for example, Northern blots, RT-PCR, arrays including microarrays and sequencing including high-throughput sequencing methods. Optionally, a reverse transcriptase reaction is carried out and the targeted sequence is then amplified using standard PCR.

Quantitative PCR (qPCR) or real time PCR (RT-PCR) is useful for determining relative expression levels, when compared to a control. Quantitative PCR techniques and platforms are known in the art and are commercially available (see, e.g., the qPCR Symposium website, available at qpersymposium.com). Nucleic acid arrays are also useful for detecting nucleic acid expression. Customizable arrays are available from, e.g., Affymatrix (Santa Clara, Calif.).

Optionally, methods for detecting RNA include sequencing methods. RNA sequencing are known and can be performed with a variety of platforms including, but not limited to, platforms provided by Illumina, Inc., (La Jolla, Calif.) or Life Technologies (Carlsbad, Calif.). See, e.g., Wang, et al., Nat Rev Genet. 10(1):57-63 (2009); and Martin, Nat Rev Genet. 12(10):671-82 (2011). Optionally, methods for detecting RNA including miRNA include microarray methods, which are known and can be performed with a variety of platforms including, but not limited to, platforms provided by Ambion, Inc., (Austin, Tex.) and Life Technologies (Carlsbad, Calif.).

Optionally, the miRNA molecules, e.g., SEQ ID NOs:1-57, are detected using one or more probes, which can be referred to herein as miRNA probes. Thus, optionally, the provided methods include contacting a sample, e.g., a biological sample, with one or more probes capable of binding to one or more of SEQ ID NOs:1-57 or any combination of SEQ ID NOs:1-57. Optionally, the probes are labeled. The miRNA probes can be DNA, RNA, nucleotide analogs, peptide nucleic acids (PNAs), or any combination of DNA, RNA, nucleotide analogs, and PNAs. The provided probes can be complementary to one or more nucleic acid residues of SEQ ID NOs:1-57. By way of example, the probes can be of 10, 11, 12, 13, 14, 15, 16, 17, 18, 19, 20, 21, 22, 23, 24, or 25 residues in length. Optionally, the probes are 25, 30, 35, 40, 45, 50, 55, 60, 65, 70, 75, 80, 85, or any length between 25-85, inclusive, residues in length. The provided probes can be complementary to at least 10 nucleic acid residues of SEQ ID NOs:1-57. Thus, the provided probes are complementary to or are at least complementary to 10, 11, 12, 13, 14, 15, 16, 17, 18, 19, 20, 21, 22, 23, 24, 25, 30, 35, 40, 45, 50, 55, 60, 65, 70, 75, 80, or 85 nucleic acid residues of the miRNA. Optionally, the nucleic acid residues of the miRNA to which the probes bind are contiguous. Optionally, the provided probes are fully complementary to the sequences of SEQ ID NOs:1-57. Thus, the provided probes can be the same as or nearly the same as the miRNA gene and complementary to the processed miRNA, i.e., mature form, or its precursors, e.g., the pri- or pre-form. As discussed above, it is contemplated that miRNA probes may be almost fully complementary (1, 2, 3, 4, 5, 6, 7, 8, 9, 10 base-pair mismatches or fewer) or fully complementary to any miRNA sequence or set of sequences that is targeted.

The disclosed methods involve comparing the levels or activity of a biomarker from a test sample to a control sample. A control sample or value refers to a sample that serves as a reference, usually a known reference, for comparison to a test sample. For example, a test sample can be taken from a patient suspected of having GWI and compared to samples from a known GWI subject or a known normal (healthy, without GWI) subject. A control can also represent an average value gathered from a population of similar individuals, e.g., GWI patients or healthy individuals with a similar medical background, same age, weight, etc. A control value can also be obtained from the same individual, e.g., from an earlier-obtained sample, prior to disease or prior to treatment. One of skill will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. Control samples may or may not be run in parallel with test samples. Optionally, the control value is a known value to which the test results are compared.

Unless clearly indicated to the contrary, the terms higher, increases, elevates, or elevation as used herein refer to increases above a normal, healthy control level or value. One of skill in the art will recognize that, if the biomarker is increased over a normal, healthy control value or sample(s), it is likely the same as, about the same as, or further increased than the same biomarker level of a subject with GWI. Similarly, unless clearly indicated to the contrary, the terms low, lower, reduces, or reduction as used herein refer to a decrease below normal healthy control levels as described above. If a biomarker is decreased in a subject as compared to a normal, healthy control, the biomarker level is likely the same as, about the same as, or lower than a sample from an individual with GWI.

The terms comparing, correlating and associated, in reference to determination of a risk factor for GWI, refers to comparing the presence or amount of the risk factor in an individual to its presence or amount in persons known to suffer from, or known to be at risk for GWI, or in persons known to be free of GWI, and assigning an increased or decreased probability of having/developing GWI to an individual based on the assay result(s).

Also provided are kits for carrying out the described methods. Provided is a kit comprising a binding agent capable of binding to a substance within a biological sample from a subject that has or is at risk for developing Gulf War Illness, wherein said substance is (i) one or more nucleic acid sequences selected from the group consisting of a miR-141 sequence, a miR-200b sequence, a miR-223 sequence, a miR-130a sequence, a miR-155 sequence, a miR-20a sequence, a miR-10a sequence, and any combination thereof, or (ii) one or more nucleic acid amplification products selected from the group consisting of a miR-141 amplification product, a miR-200b amplification product, a miR-223 amplification product, a miR-130a amplification product, a miR-155 amplification product, a miR-20a amplification product, a miR-10a amplification product, or any combination thereof; and a detecting reagent or a detecting apparatus capable of detecting binding of said binding agent to said substance. Optionally, the binding agent is a probe capable of binding the nucleic acid sequence comprising miR-141, miR-200b, miR-223, miR-130a, miR-155, miR-20a, or miR-10a or the nucleic acid amplification product of miR-141, miR-200b, miR-223, miR-130a, miR-155, miR-20a, or miR-10a. Optionally, the probe is a labeled probe. Optionally, the kits include a detecting apparatus, wherein the detecting apparatus is an apparatus for performing a Northern blot analysis, an RT-PCR, a microarray analysis or a sequencing analysis. Optionally, the kits include a control biological sample.

Provided are kits for determining a level of SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and/or 57 or a combination thereof. The provided kits include components for assessing SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and/or 57 expression comprising, e.g., a nucleic acid capable of detecting SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and/or 57, optionally labeled. Thus, the provided kits may include a binding agent capable of binding to a substance within a biological sample from a subject that has or is at risk for developing GWI, wherein said substance is (i) a nucleic acid sequence comprising SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and/or 57 sequence, or (ii) a nucleic acid amplification product of SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and/or 57. Optionally, the binding kits further include a detecting reagent or a detecting apparatus capable of detecting binding of said binding agent to said substance. Optionally, the detecting reagent is a label on the probe. Optionally, the detecting apparatus is an apparatus for performing Northern blot analysis, RT-PCR, microarray analysis or sequencing analysis. The binding agent can be a probe capable of binding the nucleic acid sequence comprising SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and/or 57 or the nucleic acid amplification product of SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and/or 57. Optionally, the probes are labeled probes. The probes can be complementary to at least 10 nucleic acid residues of SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and/or 57. Optionally, the probes are fully complementary to SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and/or 57. Optionally, the nucleic acid residues in SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and/or 57 to which the probe binds are contiguous. As discussed herein, the probes can be 10, 11, 12, 13, 14, 15, 16, 17, 18, 19, 20, 21, 22, 23, 24, or 25 residues in length. Optionally, the probe is 25, 30, 35, 40, 45, 50, 55, 60, 65, 70, 75, 80, or 85 residues in length. As discussed above, the provided probes can be designed to bind to the pri-, pre-, or mature forms of SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and/or 57. The kit can further include assay containers (tubes), buffers, or enzymes necessary for carrying out the detection assay.

Optionally, the kit includes components to examine more than one GWI marker. For example, the kit can include marker detection agents, such as marker specific primers or probes attached to an addressable array. Kits can also include components for comparing results such as a suitable control sample, for example a positive (with GWI) and/or negative (healthy, normal) control. The kit can also include a collection device for collecting and/or holding the sample from the subject. The collection device can include a sterile swab or needle (for collecting blood), and/or a sterile tube (e.g., for holding the swab or a bodily fluid sample). Optionally, the provided kits include instructions for use.

As used throughout, a subject can be any age and either sex. As used herein, patient, individual and subject may be used interchangeably and these terms are not intended to be limiting. That is, an individual described as a patient does not necessarily have a given disease, but may be merely seeking medical advice. The terms patient or subject include human and veterinary subjects.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of embodiments have been described. Nevertheless, it will be understood that various modifications may be made. Accordingly, other embodiments are within the scope of the claims below.

### EXAMPLE

### Example 1

### miRNA in Gulf War Illness (GWI)

In this example, exosomes and their miRNAs (small noncoding, inhibitor RNAs) and protein contents in cerebrospinal fluid (CSF) and plasma of GWI and sedentary control (SC) subjects were examined to determine their potential role in the exertional exhaustion and neural abnormalities that characterize GWI.

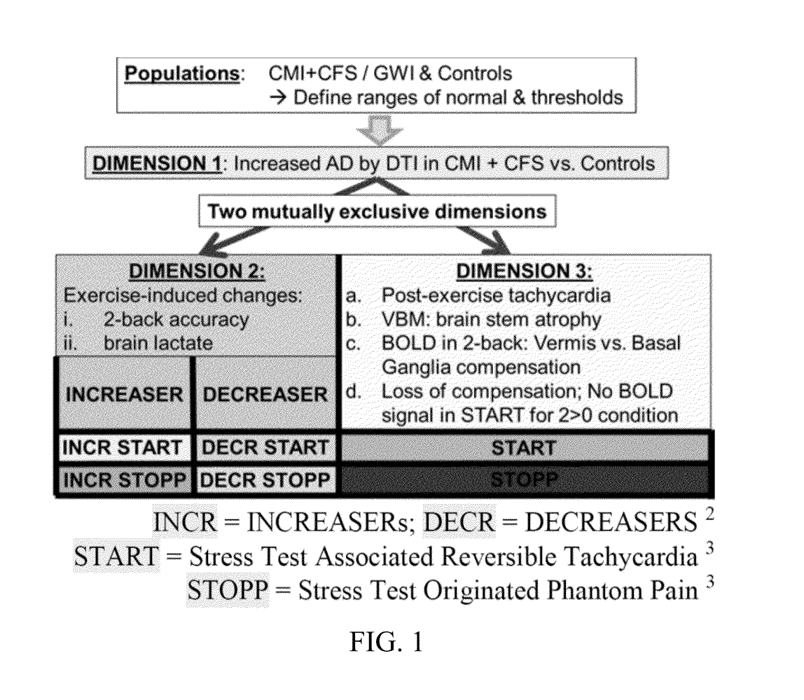

This example describes determination of the molecular mechanisms underlying GWI. Exosome miRNAs (small noncoding, inhibitor RNAs) and protein constituents in cerebrospinal fluid (CSF) and plasma may have a potential role in exertional exhaustion, cognitive dysfunction, migraine and other neurological neural abnormalities in GWI. Further, the objective magnetic resonance imaging (MRI) and exercise-induced cognitive and autonomic changes that discriminate GWI from sedentary control (SC) subjects (Table 1) was used as a framework to (i) explain symptom heterogeneity in GWI; (ii) rationalize subjective GWI criteria; 6-8 and (iii) identify specific neural pathophysiological mechanisms for objectively-defined phenotypes (FIG. 1). Table 2 is an outline of the clinical exercise study used to obtain specimens.

TABLE 1
GWI phenotypes defined by three objective “dimensions”
GWI vs. SC DIMENSION 1. Diffusion tensor imaging (DTI) showed
increased white matter axial diffusivity (AD) that separated
GWI (n = 31) from SC (n = 10). AD was most significantly
elevated in the right inferior frontal orbital fasciculus that
connects major brain regions associated with GWI symptomatology.

| | | GWI DIMENSION 2. | |
|---|---|---|---|
| | | INCREASERs | DECREASERs |
| GWI Phenotyping | | (INCR) | (DECR) |
| | START | 4[2] | 6[3] |
| GWI Dimension 3. | STOPP | 8[4] | 10[5] |

TABLE 2
Outline of clinical exercise study used to obtain specimens

| | | | | | |
|---|---|---|---|---|---|
| Screen - | Day 1 MRI | Day 1 Exercise | Day 2 MRI | Lumbar | Follow |
| baseline | & | Day 2 Exercise | & | Puncture | Fatigue, |
| status | Cognitive | (Exercise- | Cognitive | | Activity |
| | Test | induced fatigue) | Test | | |

Exercise-induces cognitive and autonomic dysfunction

Dimension 1:

GWI had loss of white matter integrity compared to SC. Axonopathy was revealed by significantly higher axial diffusivity (AD) by diffusion tensor imaging (DTI) in the right inferior frontal orbital fasciculus (rIFOF) that connects major brain regions associated with GWI symptomatology. Thus, increased AD separated GWI from SC and potentially from psychiatric conditions.

Dimension 2:

Exercise caused changes in cognition during the 2-back working memory task in GWI. Two phenotypes were found based on pre- and post-exercise accuracy and brain lactate levels measured by Mill molecular spectroscopy. INCR and DECR had comparable accuracies on the 2-back working memory task before exercise. However, exercise caused accuracies to increase in INCR and decrease in DECR. Before exercise, INCR had significantly lower brain lactate than DECR. After exercise, lactate increased in INCR to the range of the DECR group. INCR and DECR phenotypes were distinguished by stressor-induced changes in cognition and brain energy metabolism.

Dimension 3:

Brain stem atrophy, postexercise postural tachycardia, and exercise—induced changes in patterns of blood oxygenation level dependent (BOLD) flow during the 2-back task defined a different pair of phenotypes. START and STOPP phenotypes were differentiated by voxel based morphometry (VBM) and BOLD (blood oxygenation level dependent level) activation of specific brain regions during cognitive testing before and after the exercise stressor. START (Stress Test Activated Reversible Tachycardia) subjects had (i) exercise-induced postural tachycardia and diastolic hypertension; (ii) atrophy of the superior cerebellar peduncle and brain stem; (iii) BOLD activation of the midline cerebellar vermis for cognitive compensation before exercise; (iv) loss of this compensation after exercise; (v) exercise-induced activation of the default mode network (DMN; “wandering mind”) during the simple 0-back stimulus-response task; and (vi) inability to recruit additional brain regions during the more demanding 2-back working memory task after exercise (loss of cognitive reserve). STOPP (Stress Test Originated Phantom Perception) subjects were defined by (a) increased activation of the insula and other somatosensory regions by BOLD that indicated increased perceptions of pain and interoceptive input. (b) The basal ganglia were recruited for cognitive compensation, but (c) exercise abolished this compensatory activation. (d) During the 0- and 2-back tasks, STOPP activated more DMN regions than SC indicating disruption of attention network activities, and (e) recruited many additional brain regions for cognitive compensation after exercise.

DIMENSIONS 2 and 3 were mutually exclusive criteria that defined four cross-referenced phenotypes.

Exosomes are membrane-bound microvesicles that are released from cells and mediate cell-to-cell transfer of short (22-25 nucleotides), noncoding, inhibitory, RNAs (miRNA), genomic DNA, proteins and membrane lipids. The exosome surface has CD9, CD63, CD81 and other ligands that bind to integrins and other receptors on endothelium and other target cells. The exosome and target cell membranes fuse and miRNAs are released into the cytoplasm. Proinflammatory roles include transport of transforming growth factor-0, and secretion of nitric oxide, reactive oxidant species, IL-1β and IL-18. Exosomes may act as circulating hormone- or virus-like inflammasomes to transmit injury signals to organs such as brain and choroid plexus. Pre- and post-synaptic neurons and glia have active exosome systems that contribute to neural plasticity, but that also transport amyloid, tau and prion proteins and rabies viruses.

MiRNAs are products of short genes that are scattered throughout the genome. They bind to precise sequences in selected mRNAs. This prevents translation of the target mRNA and prevents protein synthesis. Over 1500 human miRNAs regulate about 50% of known proteins in cell-specific fashion. Exosome miRNAs may initiate, expand, and maintain dysfunctional phenotypes by preventing the translation and expression of critical proteins in targeted cells. The miRNAs may be biomarkers of aging, mild cognitive impairment (miR-132, 134), Alzheimer's (miR-125b, 146a, let-7), multiple sclerosis, cardiomyopathy (miR-133a), and other diseases. Ten human miRNAs regulate about 80% of the 242 presynaptic and post-synaptic proteins, with mir-515, -506, -154, -548, and mir-17 each regulating the most pre- and postsynaptic proteins. As many as 133 miRNAs were unique to CSF when pooled samples were assessed by next-generation RNA sequencing and miRDeep2 prediction software. However the yield is low which necessitates extra steps for small RNA purification, use of as much as 2 ml CSF from individual subjects, and pooling of samples from multiple subjects. Exosome RNA, protein, and lipid components can be purified from fresh and −80° C. frozen CSF and plasma.

Exertional exhaustion is a key finding in CFS and GWI. When subjects do more physical or cognitive activity than usual, they develop a flare of illness with onset after 0 to 24 hours that is not relieved by sleep and may last several days. One of our outcomes was accuracy on the 2-back working memory task. Exercise caused half of GWI to have higher accuracy (INCREASERs, INCR), and half to have lower accuracy (DECREASERs, DECR) on Day 2. Controls had a ceiling effect (>80% accuracy both days). Exosomes released from peripheral sites into blood after exercise may circulate to the brain to induce changes in protein expression that leads to cognitive and brain dysfunction.

It is proposed herein that exercise stimulates exosome release from muscle, heart or lungs into blood with circulation to brain endothelial, astrocyte, or choroid plexus epithelial cells. Exosome fusion and miRNA uptake into these cells inhibits protein translation and disrupts neuro-vascular autoregulation as seen by postural tachycardia and BOLD during cognition, and the neuron-astrocyte lactate shuttle that account for exercise-induced increases in brain lactate in the INCR subset (FIG. 1). Cognitive dysfunction may be related to increased miR-132 that blocks translation of hippocampal acetylcholinesterase mRNA. Sarin and pyridostigmine bromide (PB) are acetylcholinesterase inhibitors that have been implicated in the initiation of GWI pathogenesis. Thus, it is proposed that CSF exosomes from GWI and CFS subjects have unique quantitative and qualitative patterns of miRNA expression compared to SC, and that exercise alters these patterns.

GWI subjects can be divided into subsets based on exercise-induced changes in postural tachycardia, brain stem atrophy, brain blood flow, brain lactate levels, and exercise-induced changes in accuracy on cognitive testing and miRNA levels. GWI were distinguished from sedentary controls (SC) using hsa-miR-141-3p, hsa-miR-200b-3p, hsa-miR-223-3p, hsa-miR-130a-3p, hsa-miR-155-5p, hsa-miR-20a-3p, and hsa-miR-10a-3p. Table 4 shows the results of quantitative real time PCR from subjects normalized to RNU6-2, SNORD 61, SNORD 68, SNORD 92, and SNORD 96A and also normalized to mean of sedentary controls (SC, n=4). Statistical analysis involved 2-tailed unpaired Student's t-test outcomes, uncorrected and n=4 per group.

TABLE 4
qRT-PCR Analysis of miRNA

| Upper vs. | SC | SC | START | SC | SC | INCR | SC |
|---|---|---|---|---|---|---|---|
| Lower Row | START | STOPP | STOPP | INCR | DECR | DECR | GWI |
| hsa-miR-141-3p | SC > START | SC > STOPP | | SC > INCR | | | SC > GWI |
| hsa-miR-200b-3p | SC > START | SC > STOPP | | SC > INCR | SC > DECR | | SC > GWI |
| hsa-223-3p | | SC > STOPP | | SC > INCR | | | SC > GWI |
| hsa-130a-3p | SC > START | | | SC > INCR | SC > DECR | | SC > GWI |
| hsa-miR-155-5p | | SC > STOPP | | | | | SC > GWI |
| hsa-miR20a-3p | | | | SC > INCR | | | SC > GWI |
| hsa-miR-10a-5p | SC > START | | STOPP > START | | SC > DECR | | SC > GWI |
| hsa-miR-191-5p | | | START > STOPP | | | | |
| hsa-miR-92b-5p | | | STOPP > START | | | | |
| hsa-miR-483-5p | | | STOPP > START | | | | |
| hsa-miR-200a-5p | | | STOPP > START | | | | |
| hsa-miR-136-5p | SC > START | | STOPP > START | | | | |
| hsa-miR-129-2-3p | SC > START | | STOPP > START | | | | |
| hsa-miR-383 | SC > START | | STOPP > START | | | | |
| hsa-miR-432-5p | SC > START | | STOPP > START | | | | |
| hsa-miR-373-3p | SC > START | | STOPP > START | | | | |
| hsa-miR-200c-3p | SC > START | | STOPP > START | | | | |
| hsa-miR-196a-5p | SC > START | | | | SC > DECR | | |
| hsa-miR-99a-3p | SC > START | | | | SC > DECR | | |
| hsa-186-3p | SC > START | | | | | | |
| hsa-miR-142-5p | START > SC | | | INCR > SC | | INCR > DECR | |
| hsa-miR-21-5p | | | | | | DECR > INCR | |
| hsa-miR-126-3p | | SC > STOPP | | | | | |
| hsa-miR-18b-3p | | STOPP > SC | | | | | |
| hsa-miR-187-3p | | STOPP > SC | | | | | |

A summary of the results is shown in Table 5.

TABLE 5

| | | INCREASERS | DECREASERS |
|---|---|---|---|
| | Two mutually exclusive, independent sets of MRI, | Significantly | High baseline |
| | cognitive test, | lower preexercise | brain lactate |
| | exercise-induced postural heart rate changes, and | brain lactate | levels. |
| | exercise-induced changes in brain blood flow and | levels by molecular | No change in |
| | lactate levels allowed the GWI subjects to be | spectroscopy. | high levels |
| | divided into either the START - STOPP or | Significantly | after exercise. |
| | INCREASER - DECREASER subtypes. | increased brain | Significant |
| | | lactate levels | decrease in |
| | | after exercise. | cognitive |
| | | Significantly | accuracy after |
| | | higher cognitive | exercise. |
| | | test accuracy | DECREASER > |
| | | after exercise. | INCREASER for |
| | | INCREASER > | hsa-miR-21-5p |
| | | DECREASER | in buffy coat |
| | | for hsa-miR-142-5p | |
| | | in buffy coat. | |
| START | Post-exercise postural tachycardia (heart rate | START- | START- |
| | increases >30 beats per minute when standing up | INCREASERS | DECREASERS |
| | from a seated position. | | |
| | Decreased volume of brain stem regions by MRI. | | |
| | Loss of significant changes in brain blood flow | | |
| | during the cognitive testing after exercise | | |
| | indicating exercise-induced dysfunction and loss | | |
| | of cognitive compensation. | | |
| | START > STOPP for hsa-miR-191-5p level in | | |
| | buffy coat (white blood cells). | | |
| | START > STOPP in cerebrospinal fluid by next | | |
| | generation | | |
| | sequencing: MIR10A; MIR10B; MIR99B; | | |
| | MIR22, MIR22HG; MIR30A; MIR182; MIRLET7C; MIR30D; | | |
| | MIR27B; MIR27A; MIR17, MIR17HG, MIR18A, MIR19A, | | |
| | MIR19B1, MIR20A, MIR92A1; MIR4763, MIRLET7A3, | | |
| | MIRLET7B, MIRLET7BHG; MIRLET7D; MIR93; MIR183; | | |
| | MIR186; MIR486; and MIRLET7A2 | | |
| STOPP | No postural tachycardia at any time. | STOPP- | STOPP- |
| | Normal brain stem by MRI. | INCREASERS | DECREASERS |
| | Increased brain blood flow after exercise indicating | | |
| | recruitment of additional brain regions to complete | | |
| | the cognitive test. | | |
| | STOPP > START for hsa-miR-10a-5p, hsa-miR-92b-5p, | | |
| | hsa-miR-483-5p, hsa-miR-200a-5p, hsa-miR-136-5p, | | |
| | hsa-miR-129-2-3p, hsamiR-383, hsa-miR-432-5p, | | |
| | hsa-miR-373-3p, and hsa-miR-200c-3p in | | |
| | buffy coat (white blood cells). | | |
| | STOPP > START in cerebrospinal fluid by next | | |
| | generation | | |
| | sequencing: MIR142, MIR204 and MIR1298 | | |

As expected for CSF, quantitative real time reverse transcriptase polymerase chain reaction (Q-PCR) identified miR-92a, but no detectable miR-15 or mi-U6. Preliminary next generation RNA sequencing (NGS) studies were initiated. We pooled RNA from pairs of 0.5 ml CSF specimens from 6 START, 6 STOPP and 6 SC subjects to give 3 RNA samples per group. High performance liquid chromatography confirmed adequate small RNA extraction. Small RNA was amplified for 15 cycles with miRNA primers, and sequenced (HiSeq2000, Illumina) in a protocol highly similar to previous CSF studies (Otogenetics Corp.).

Visual inspection revealed 10 trends (Table 6). As found previously by our Q-PCR, U6 was not detected, so it could not be used for normalization. Instead, XLOC_012808 (MIR216B) and XLOC_015585 (MIR191) were approximately equivalent across the 3 samples. 10 miRNA were found in SC only. XLOC_009120 was higher in START and STOPP than SC. START had 10 miRNAs that were higher than in SC or STOPP. Only START expressed 7 of the miRNAs. STOPP was highest for 2. The average reads per million for miRNA was 13,825 for SC, 74,296 for START and 64,217 for STOPP. These are unlikely to be the only miRNAs, since controls had 238 sequences that were not matched to miRNA genes (average reads=6,514,796). START had 118 sequences (10,319,139 average reads) and STOPP 221 (average 10,519,848 reads). Informatics BLAST and other searches based on the RNA sequences and genome nucleotide locations will be needed to determine if these are previously undetected miRNAs, small nucleolar or other functional small RNA species, or artifacts. Short sequences matching protein genes were also identified at low read rates in SC (n=125, average=419), START (n=87, average=328), and STOPP (n=168, average=814). These may be degraded mRNAs rather than gene specific miRNAs. The results are shown in Tables 6, 7, 8, and 9.

TABLE 6
Preliminary miRNA expression data for CSF from SC before
exercise and post-exercise START and STOPP subjects
(reads per million). Cells in bold font have <3-
fold differences and were considered equivalent.

| SC | START | STOPP | gene_id |
|---|---|---|---|
| 11,814 | 7,526 | 8,859 | XLOC_012808 |
| 12,310 | 20,966 | 8,442 | XLOC_015585 |
| 28,819 | 0 | 0 | XLOC_021068 |
| 21,524 | 0 | 0 | XLOC_009503 |
| 4,843 | 0 | 0 | XLOC_008863 |
| 3,739 | 0 | 0 | XLOC_015495 |
| 2,719 | 0 | 0 | XLOC_021767 |
| 2,522 | 0 | 0 | XLOC_004943 |
| 1,769 | 0 | 0 | XLOC_020633 |
| 1,204 | 0 | 0 | XLOC_017297 |
| 534 | 0 | 0 | XLOC_006577 |
| 104 | 0 | 0 | XLOC_014000 |
| 9,073 | 9,848 | 0 | XLOC_012418 |
| 13,343 | 0 | 10,404 | XLOC_022740 |
| 141,932 | 636,042 | 651,476 | XLOC_009120 |
| 9,927 | 49,111 | 6,592 | XLOC_010971 |
| 7,291 | 65,018 | 17,226 | XLOC_009293 |
| 38,894 | 149,514 | 529 | XLOC_009723 |
| 17,442 | 305,613 | 0 | XLOC_018826 |
| 6,574 | 23,448 | 0 | XLOC_005908 |
| 4,436 | 20,463 | 0 | XLOC_022052 |
| 2,522 | 23,267 | 0 | XLOC_014009 |
| 1,461 | 42,345 | 0 | XLOC_021175 |
| 800 | 13,036 | 0 | XLOC_022320 |
| 0 | 20,517 | 0 | XLOC_011370 |
| 0 | 15,129 | 0 | XLOC_001596 |
| 0 | 12,505 | 0 | XLOC_022313 |
| 0 | 10,832 | 0 | XLOC_021177 |
| 0 | 8,946 | 0 | XLOC_021836 |
| 0 | 8,128 | 0 | XLOC_021069 |
| 0 | 3,753 | 0 | XLOC_004562 |
| 1 | 36,575 | 3,890 | XLOC_014525 |
| 0 | 141,887 | 56,445 | XLOC_022625 |
| 0 | 0 | 4,487 | XLOC_023446 |
| 0 | 0 | 2,238 | XLOC_009779 |

TABLE 7
Normalized data sorted. Cerebrospinal fluid assessed
in duplicate by next generation sequencing.

| test id | Gene(normalized; log2) | Control | START | STOPP |
|---|---|---|---|---|
| XLOC_001596 | MIR186 | 0 | 6.525312 | 0 |
| XLOC_004562 | MIRLET7A2 | 0 | 1.618837 | 0 |
| XLOC_004943 | MIRLET7I | 1.087832 | 0 | 0 |
| XLOC_005908 | MIR17, MIR17HG, MIR18A, MIR19A, | 2.835602 | 10.1136 | 0 |
| | MIR19B1, MIR20A, MIR92A1 | | | |
| XLOC_006577 | MIR203 | 0.230182 | 0 | 0 |
| XLOC_006911 | MIR3545 | 0 | 0 | 0 |
| XLOC_008863 | MIR451B | 2.089025 | 0 | 0 |
| XLOC_009120 | MIR21 | 61.21893 | 274.3413 | 268.5147 |
| XLOC_009293 | MIR22, MIR22HG | 3.144733 | 28.04372 | 7.099889 |
| XLOC_009490 | MIR451A | 0.001946 | 0 | 0 |
| XLOC_009503 | MIR3184 | 9.283704 | 0 | 0 |
| XLOC_009723 | MIR10A | 16.77585 | 64.48924 | 0.217913 |
| XLOC_009779 | MIR142 | 0 | 0 | 0.922375 |
| XLOC_010971 | MIR99B | 4.281976 | 21.18284 | 2.716894 |
| XLOC_011370 | MIR27A | 0 | 8.849402 | 0 |
| XLOC_012418 | MIR10B | 3.913621 | 4.24751 | 0 |
| XLOC_012808 | MIR216B | 5.095661 | 3.246112 | 3.651452 |
| XLOC_014000 | MIR3648 | 0.044853 | 0 | 0 |
| XLOC_014009 | MIRLET7C | 1.087832 | 10.03544 | 0 |
| XLOC_014525 | MIR4763, MIRLET7A3, MIRLET7B, | 0.000432 | 15.77576 | 1.603516 |
| | MIRLET7BHG | | | |
| XLOC_015495 | MIR4792 | 1.612749 | 0 | 0 |
| XLOC_015585 | MIR191 | 5.309771 | 9.043111 | 3.479592 |
| XLOC_017297 | MIR143, MIR143HG, MIR145 | 0.519157 | 0 | 0 |
| XLOC_018826 | MIR30A | 7.523076 | 131.8188 | 0 |
| XLOC_020633 | MIR335 | 0.763004 | 0 | 0 |
| XLOC_021068 | MIR25 | 12.43025 | 0 | 0 |
| XLOC_021069 | MIR93 | 0 | 3.505723 | 0 |
| XLOC_021175 | MIR182 | 0.63027 | 18.26432 | 0 |
| XLOC_021177 | MIR183 | 0 | 4.672271 | 0 |
| XLOC_021767 | MIR320A | 1.172917 | 0 | 0 |
| XLOC_021836 | MIR486 | 0 | 3.858651 | 0 |
| XLOC_022052 | MIR30D | 1.913497 | 8.826176 | 0 |
| XLOC_022313 | MIRLET7D | 0 | 5.393621 | 0 |
| XLOC_022320 | MIR27B | 0.34518 | 5.622568 | 0 |
| XLOC_022625 | MIR31 | 0 | 61.19952 | 23.2647 |
| XLOC_022740 | MIR204 | 5.75533 | 0 | 4.287965 |
| XLOC_023446 | MIR1298 | 0 | 0 | 1.849553 |

TABLE 8
Normalized data sorted and ranked.
Cerebrospinal fluid assessed in duplicate by next generation sequencing. Four-fold
changes were considered significant for this preliminary study of n = 2 per group.
0 indicates transcript not detected in 2 ml of cerebrospinal fluid.

| test_id | Gene (normalized; log2) | SC | START | STOPP | Ranking |
|---|---|---|---|---|---|
| XLOC_012808 | MIR216B | 5 | 3 | 4 | Equivalent |
| XLOC_015585 | MIR191 | 5 | 9 | 3 | Equivalent |
| XLOC_021068 | MIR25 | 12 | 0 | 0 | SC > START = STOPP |
| XLOC_009503 | MIR3184 | 9 | 0 | 0 | SC > START = STOPP |
| XLOC_008863 | MIR451B | 2 | 0 | 0 | SC > START = STOPP |
| XLOC_015495 | MIR4792 | 2 | 0 | 0 | SC > START = STOPP |
| XLOC_021767 | MIR320A | 1 | 0 | 0 | SC > START = STOPP |
| XLOC_004943 | MIRLET7I | 1 | 0 | 0 | SC > START = STOPP |
| XLOC_020633 | MIR335 | 1 | 0 | 0 | SC > START = STOPP |
| XLOC_017297 | MIR143, MIR143HG, | 1 | 0 | 0 | SC > START = STOPP |
| | MIR145 | | | | |
| XLOC_009723 | MIR10A | 17 | 64 | 0 | SC = START > STOPP = 0 |
| XLOC_012418 | MIR10B | 4 | 4 | 0 | SC = START > STOPP = 0 |
| XLOC_022740 | MIR204 | 6 | 0 | 4 | SC = STOPP > START = 0 |
| XLOC_022625 | MIR31 | 0 | 61 | 23 | START = STOPP > SC = 0 |
| XLOC_009120 | MIR21 | 61 | 274 | 269 | START = STOPP > SC |
| XLOC_010971 | MIR99B | 4 | 21 | 3 | START > SC = STOPP |
| XLOC_009293 | MIR22, MIR22HG | 3 | 28 | 7 | START > SC = STOPP |
| XLOC_018826 | MIR30A | 8 | 132 | 0 | START > SC > STOPP = 0 |
| XLOC_021175 | MIR182 | 1 | 18 | 0 | START > SC > STOPP = 0 |
| XLOC_005908 | MIR17, MIR17HG, | 3 | 10 | 0 | START > SC > STOPP = 0 |
| | MIR18A, MIR19A, | | | | |
| | MIR19B1, | | | | |
| | MIR20A, | | | | |
| | MIR92A1 | | | | |
| XLOC_014009 | MIRLET7C | 1 | 10 | 0 | START > SC > STOPP = 0 |
| XLOC_022052 | MIR30D | 2 | 9 | 0 | START > SC > STOPP = 0 |
| XLOC_022320 | MIR27B | 0 | 6 | 0 | START > SC > STOPP = 0 |
| XLOC_014525 | MIR4763, | 0 | 16 | 2 | START > STOPP > SC = 0 |
| | MIRLET7A3, | | | | |
| | MIRLET7B, | | | | |
| | MIRLET7BHG | | | | |
| XLOC_011370 | MIR27A | 0 | 9 | 0 | START > SC = STOPP = 0 |
| XLOC_001596 | MIR186 | 0 | 7 | 0 | START > SC = STOPP = 0 |
| XLOC_022313 | MIRLET7D | 0 | 5 | 0 | START > SC = STOPP = 0 |
| XLOC_021177 | MIR183 | 0 | 5 | 0 | START > SC = STOPP = 0 |
| XLOC_021836 | MIR486 | 0 | 4 | 0 | START > SC = STOPP = 0 |
| XLOC_021069 | MIR93 | 0 | 4 | 0 | START > SC = STOPP = 0 |
| XLOC_004562 | MIRLET7A2 | 0 | 2 | 0 | START > SC = STOPP = 0 |
| XLOC_009779 | MIR142 | 0 | 0 | 1 | STOPP > SC = START = 0 |
| XLOC_023446 | MIR1298 | 0 | 0 | 2 | STOPP > SC = START = 0 |

TABLE 9
Selected group (putative biosignature)

| | | | | | |
|---|---|---|---|---|---|
| XLOC_009723 | MIR10A | 17 | 64 | 0 | SC = START > STOPP = 0 |
| XLOC_012418 | MIR10B | 4 | 4 | 0 | SC = START > STOPP = 0 |
| XLOC_010971 | MIR99B | 4 | 21 | 3 | START > SC = STOPP |
| XLOC_009293 | MIR22, MIR22HG | 3 | 28 | 7 | START > SC = STOPP |
| XLOC_018826 | MIR30A | 8 | 132 | 0 | START > SC > STOPP = 0 |
| XLOC_021175 | MIR182 | 1 | 18 | 0 | START > SC > STOPP = 0 |
| XLOC_005908 | MIR17, | 3 | 10 | 0 | START > SC > STOPP = 0 |
| | MIR17HG, | | | | |
| | MIR18A, | | | | |
| | MIR19A, | | | | |
| | MIR19B1, | | | | |
| | MIR20A, | | | | |
| | MIR92A1 | | | | |
| XLOC_014009 | MIRLET7C | 1 | 10 | 0 | START > SC > STOPP = 0 |
| XLOC_022052 | MIR30D | 2 | 9 | 0 | START > SC > STOPP = 0 |
| XLOC_022320 | MIR27B | 0 | 6 | 0 | START > SC > STOPP = 0 |
| XLOC_014525 | MIR4763, | 0 | 16 | 2 | START > STOPP > SC = 0 |
| | MIRLET7A3, | | | | |
| | MIRLET7B, | | | | |
| | MIRLET7BHG | | | | |
| XLOC_011370 | MIR27A | 0 | 9 | 0 | START > SC = STOPP = 0 |
| XLOC_001596 | MIR186 | 0 | 7 | 0 | START > SC = STOPP = 0 |
| XLOC_022313 | MIRLET7D | 0 | 5 | 0 | START > SC = STOPP = 0 |
| XLOC_021177 | MIR183 | 0 | 5 | 0 | START > SC = STOPP = 0 |
| XLOC_021836 | MIR486 | 0 | 4 | 0 | START > SC = STOPP = 0 |
| XLOC_021069 | MIR93 | 0 | 4 | 0 | START > SC = STOPP = 0 |
| XLOC_004562 | MIRLET7A2 | 0 | 2 | 0 | START > SC = STOPP = 0 |

The patterns of miRNA (patterns of those present and absent) were highly different between the pre-exercise SC and post-exercise GWI subgroups of START and STOPP. This suggests significant differences in neuropathology between these 3 states and justifies use of the patterns to identify molecular mechanisms, disordered cellular pathways (e.g., GO terms), and GWI phenotype specific “biosignatures” for diagnostic purposes (inexpensive, rapid turnaround time, high speed Q-RT-PCR).

These results demonstrated that muscle, heart, lung or other peripheral exosomes and miRNAs may be delivered to the brain and alter function. This would be consistent with exertional exhaustion. The differences between phenotypes in Table 2 makes it plausible that changes in brain exosomes may alter the information passed between neurons as synapses, or between other brain cells.

### Example 2

### Protein and miRNA Patterns in Exosomes from CSF and the Effect of Exercise

As described herein, specific patterns of miRNAs and proteins are determined in exosomes from CSF to infer differences for pre- vs. post-exercise and GWI subsets vs. SC. Significant changes provide a mechanistic foundation to explain GWI phenotypes. Circadian changes in plasma exosome miRNAs and proteins are also detected. Post-exercise plasma exosome miRNA and protein patterns identify selective changes in expression in GWI subtypes vs. SC.

Samples & Subjects: CSF (n=132) and plasma (n=817) samples stored at −80° C. are assessed. Serial plasma samples for circadian and exercise-induced variations of exosome and miRNA quantity and content are collected from (A) SC, CFS, and GWI subjects who have two exercise sessions but no MRI studies; (B) normal males and females who have one exercise session; and (C) GWI subjects who have exercise and MM (n=29), and are classified as START-INCR, START-DECR, STOPP-INCR, and STOPP-DECR (FIG. 1). Plasma is drawn at the time of exercise on Days 1 & 2, and 3, 8 and 24 hours after each exercise session, and at comparable times on the Screening Day before exercise. Heparinized plasma is immediately centrifuged, aliquoted and frozen at −80° C. Samples with hemolysis are discarded and redrawn to prevent contamination from erythrocytes.

Instead of extracting miRNA alone, exosomes for miRNA and protein analysis are extracted using polymer-based ExoQuick kits (System Biosciences).

Standard microarray quantitative RT-PCR (Q-PCR) is performed for 380 miRNAs. Q-PCR requires 1 ml of CSF for exosome extraction and miRNA purification to obtain approximately 400 ng of total RNA. In preliminary Q-PCR based array analysis, this amount produces robust miRNA detection.

The ExoQuick precipitated exosomes are resuspended in 10 volumes of Qiazol lysis reagent and vortexed. The lysate is extracted with CHCl₃ and the aqueous phase is further enriched for miRNA using the miRNeasy kit (Qiagen). The miRNA enriched fraction is eluted in RNase free water. The miRNA is converted to cDNA using miScriptII RT kit. miRNA expression profiling is performed using miScript miRNA PCR arrays (Qiagen) with the miScript SYBRgreen PCR kit on an ABI 7900 HT Real-Time PCR system (Applied Biosystems, Foster City, Calif.). The array assays 384 miRNAs. Four normalizers are included to correct for differences in the RNA input between samples. Other extraction kits are used to detect longer RNA, genomic DNA, and systematic extraction biases.

A small set of miRNAs (<20) is identified that discriminate the various GWI phenotypes from each other and controls at both the pre- and post-exercise time points. Individual Q-PCR assays are used (Qiagen). Differences in expression between the samples are calculated using Delta-Delta C(T) method with commercial software. The data are expressed as fold up-regulation or down-regulation in miRNA expression compared to the control samples.

Exosome miRNA statistical analysis. The nature of the data lends itself to several statistical approaches. Multivariate regression can be used, as well as mean-centering with normalization for hierarchical clustering (“pvclust”) and bootstrapping (“sbfit”) in R 77 followed by random forest classification. These methods are used to assess miRNA patterns correlating with START vs. STOPP and INCR vs. DECR phenotypes, and GWI vs. SC. A general linear or mixed regression model is advantageous by setting age, gender, pre-exercise status as GWI, CFS or SC, and post-exercise status as STOPP, START or SC as variables. The advantage of these methods over Fisher's Exact Test is that independent variables such as age, gender, presence of comorbid conditions such as migraine, irritable bowel syndrome, dolorimetry measures of systemic hyperalgesia, post-exercise postural tachycardia, and individual magnetic resonance imaging (MM) modality measurements from example 1 can be included to assess potential groupings of variables that may identify more instructive and inclusive phenotypic patterns. The more concise statistical results infer causality and generate new hypotheses about disease mechanisms, selection of subsets of miRNAs for diagnosis, and the like. Smaller, sensitive and specific sets of highly discriminating target miRNAs are used for economical, high throughput, and wide dynamic range Q-PCR testing of all specimens.

Exosomes are isolated from frozen CSF 83 and plasma (ExoQuick) and enumerated. After the ExoQuick precipitated exosomes are resuspended in Qiazol lysis reagent, and the miRNA extracted into the aqueous phase by CHCl3, the non-RNA fractions are retained for protein purification. The problem faced in other proteomics applications is to remove the detergent, any guanidinium and other surfactants from the protein fraction. This is achieved by serial precipitation in butanol, isopropanol and ethanol. In each case, the organic phase solubilizes the most lipophilic and amphipathic surfactants leaving a pellet of increasingly “clean” protein. Care is taken during the resuspension steps between each organic precipitation to ensure that the entire, small protein pellet is resuspended and washed. To facilitate this, a mass spectrometry friendly detergent, and TCEP [tris(2-carboxyethyl)phosphine], a reducing agent, is resuspended in 1% RapiGestSF (Waters). These also facilitate the transfer and solubilization of membrane proteins that help identify the potential cells of origin of the exosome fraction. After ethanol precipitation, the pellet is reconstituted in 25 μL and protein concentrations are determined by Nanodrop spectroscopy. Trypsin is added (1:20) before overnight digestion at 37° C. Trifluroacetic acid (final concentration 0.5%) and acetonitrile (5%) are added, and the tryptic peptides (6 μL) separated by reversed phase liquid capillary chromatography before Orbitrap mass spectrometry.

The 1st dimension of mass spectrometry provides the number of ion peaks (“spectral counts”), mass/charge, retention times, ion peak signal intensities (“ion count”), and total ion count that allows the ion peak to be aligned. The 2nd mass spectrometry dimension is the peptide sequencing. Peptides are sequenced and aligned using Protein Information Resource peptide matching function and MASCOT software (Matrix Sciences, Boston).

Searches for posttranslational modifications are performed to identify significant oxidation in GWI but not SC. Comparisons of hierarchical clusters of peptides discovered in each phenotypic group identify shared exosome proteins, plus proteins from all of the other cells that are secreting exosomes. Differences in peptide profiles are monitored between SC and GWI, and GWI phenotypes. Proteins identified by MASCOT peptide matching software are assessed for potential cells of origin, target cells if receptors are present, GO, Reactome and KEGG pathways, and miRNAs that regulate their expression.

Sequence Listing

| | | SEQ |
|---|---|---|
| Mature miRNA | Sequence 5′-3′ | ID NO: |
| hsa-let-7a-5p | UGAGGUAGUAGGUUGUAUAGUU | 1 |
| hsa-let-7c-5p | UGAGGUAGUAGGUUGUAUGGUU | 2 |
| hsa-let-7d-5p | AGAGGUAGUAGGUUGCAUAGUU | 3 |
| hsa-let-7d-3p | CUAUACGACCUGCUGCCUUUCU | 4 |
| hsa-let-7e-5p | UGAGGUAGGAGGUUGUAUAGUU | 5 |
| hsa-miR-10a-5p | UACCCUGUAGAUCCGAAUUUGUG | 6 |
| hsa-miR-10a-3p | CAAAUUCGUAUCUAGGGGAAUA | 7 |
| hsa-miR-10b-5p | UACCCUGUAGAACCGAAUUUGUG | 8 |
| hsa-miR-10b-3p | ACAGAUUCGAUUCUAGGGGAAU | 9 |
| hsa-miR-129-2-3p | AAGCCCUUACCCCAAAAAGCAU | 10 |
| hsa-miR-129-5p | CUUUUUGCGGUCUGGGCUUGC | 11 |
| hsa-miR-130a-3p | CAGUGCAAUGUUAAAAGGGCAU | 12 |
| hsa-miR-136-5p | ACUCCAUUUGUUUUGAUGAUGGA | 13 |
| hsa-miR-141-3p | UAACACUGUCUGGUAAAGAUGG | 14 |
| hsa-miR-142-3p | UGUAGUGUUUCCUACUUUAUGGA | 15 |
| hsa-miR-142-5p | CAUAAAGUAGAAAGCACUACU | 16 |
| hsa-miR-155-5p | UUAAUGCUAAUCGUGAUAGGGGU | 17 |
| hsa-miR-155-3p | CUCCUACAUAUUAGCAUUAACA | 18 |
| hsa-miR-17-5p | CAAAGUGCUUACAGUGCAGGUAG | 19 |
| hsa-miR-182-5p | UUUGGCAAUGGUAGAACUCACACU | 20 |
| hsa-miR-182-3p | UGGUUCUAGACUUGCCAACUA | 21 |
| hsa-miR-183-5p | UAUGGCACUGGUAGAAUUCACU | 22 |
| hsa-miR-186-5p | CAAAGAAUUCUCCUUUUGGGCU | 23 |
| hsa-miR-186-3p | GCCCAAAGGUGAAUUUUUUGGG | 24 |
| hsa-miR-200a-3p | UAACACUGUCUGGUAACGAUGU | 25 |
| hsa-miR-200a-5p | CAUCUUACCGGACAGUGCUGGA | 26 |
| hsa-miR-200b-3p | UAAUACUGCCUGGUAAUGAUGA | 27 |
| hsa-miR-200c-3p | UAAUACUGCCGGGUAAUGAUGGA | 28 |
| hsa-miR-200c-5p | CGUCUUACCCAGCAGUGUUUGG | 29 |
| hsa-miR-204-5p | UUCCCUUUGUCAUCCUAUGCCU | 30 |
| hsa-miR-20a-5p | UAAAGUGCUUAUAGUGCAGGUAG | 31 |
| hsa-miR-20a-3p | ACUGCAUUAUGAGCACUUAAAG | 32 |
| hsa-miR-22-3p | AAGCUGCCAGUUGAAGAACUGU | 33 |
| hsa-miR-223-3p | UGUCAGUUUGUCAAAUACCCCA | 34 |
| hsa-miR-223-5p | CGUGUAUUUGACAAGCUGAGUU | 35 |
| hsa-miR-27a-3p | UUCACAGUGGCUAAGUUCCGC | 36 |
| hsa-miR-27a-5p | AGGGCUUAGCUGCUUGUGAGCA | 37 |
| hsa-miR-27b-3p | UUCACAGUGGCUAAGUUCUGC | 38 |
| hsa-miR-27b-5p | AGAGCUUAGCUGAUUGGUGAAC | 39 |
| hsa-miR-30a-5p | UGUAAACAUCCUCGACUGGAAG | 40 |
| hsa-miR-30d-5p | UGUAAACAUCCCCGACUGGAAG | 41 |
| hsa-miR-30d-3p | CUUUCAGUCAGAUGUUUGCUGC | 42 |
| hsa-miR-30e-5p | UGUAAACAUCCUUGACUGGAAG | 43 |
| hsa-miR-373-3p | GAAGUGCUUCGAUUUUGGGGUGU | 44 |
| hsa-miR-373-5p | ACUCAAAAUGGGGGCGCUUUCC | 45 |
| hsa-miR-383-5p | AGAUCAGAAGGUGAUUGUGGCU | 46 |
| hsa-miR-432-5p | UCUUGGAGUAGGUCAUUGGGUGG | 47 |
| hsa-miR-483-5p | AAGACGGGAGGAAAGAAGGGAG | 48 |
| hsa-miR-486-3p | CGGGGCAGCUCAGUACAGGAU | 49 |
| hsa-miR-486-5p | UCCUGUACUGAGCUGCCCCGAG | 50 |
| hsa-miR-92b-3p | UAUUGCACUCGUCCCGGCCUCC | 51 |
| hsa-miR-92b-5 | AGGGACGGGACGCGGUGCAGUG | 52 |
| hsa-miR-93-5p | CAAAGUGCUGUUCGUGCAGGUAG | 53 |
| hsa-miR-93-3p | ACUGCUGAGCUAGCACUUCCCG | 54 |
| hsa-miR-99b-5p | CACCCGUAGAACCGACCUUGCG | 55 |
| hsa-miR-4763 | AGGCAGGGGCUGGUGCUGGGCGGG | 56 |
| hsa-miR-1298 | UUCAUUCGGCUGUCCAGAUGUA | 57 |

What is claimed is:

1. A method of determining a level of one or more miRNAs in a subject that has or is at risk of developing Gulf War Illness (GWI), the method comprising the steps of:
(a) obtaining a biological sample from the subject; and
(b) determining a level of miR-141, miR-200b, miR-223, miR-130a, miR-155, miR-20a, miR-10a, or any combination thereof in the biological sample.

2. The method of claim 1, wherein the step of determining the level of miRNAs comprises performing a Northern Blot, RT-PCR, microarray analysis, or sequencing.

3. The method of claim 1, wherein the biological sample is a biological fluid.

4. The method of claim 3, wherein the biological sample is selected from the group consisting of cerebrospinal fluid, brain cells, urine, peripheral blood white cells, blood, plasma, and serum.

5. The method of claim 3, wherein the biological sample comprises natural killer cells, CD4+ cells, CD8+ cells, IL17+ cells, B lymphocytes or combinations thereof.

6. The method of claim 1, wherein the level of one or more of miR-141, miR-200b-miR-223, miR-130a, miR-155, miR-20a, or miR-10a is reduced as compared to a control.

7. The method of claim 1, further comprising determining a level of miR-10b, miR-99b, miR-22, miR-30a, miR-182, miR-LET7c, miR-30d, miR-27b, miR-27a, miR-17, miR-4763, miR-LET7d, miR-93, miR-183, miR-186, miR-486, and miR-LET7a2, or any combination thereof.

8. The method of claim 7, wherein the level of one or more of miR-10b, miR-99b, miR-22, miR-30a, miR-182, miR-LET7c, miR-30d, miR-27b, miR-27a, miR-17, miR-4763, miR-LET7d, miR-93, miR-183, miR-186, miR-486, or miR-LET7a2 is increased as compared to a control.

9. The method of claim 1, further comprising determining a level of miR-142, miR-204, miR-1298, or any combination thereof.

10. The method of claim 9, wherein the level of one or more of miR-142, miR-204, or miR-1298 is increased as compared to a control.

11. The method of claim 1, further comprising determining a level of miR-92b, miR-483, miR-200a, miR-136, miR-129, miR-383, miR-432, miR-373, miR-200c, or any combination thereof.

12. The method of claim 1, wherein the subject has GWI.

13. The method of claim 12, wherein the subject has GWI, subtype stress test activated reversible tachycardia (START).

14. The method of claim 12, wherein the subject has GWI, subtype stress test originated phantom perception (STOPP).

15. The method of claim 1, further comprising performing molecular spectroscopy, measuring exercise-induced heart rate changes, measuring exercise-induced cerebral blood flow, measuring exercise-induced lactate levels, performing magnetic resonance imaging of the brain, or combinations thereof.

16. A kit comprising
(a) a binding agent capable of binding to a substance within a biological sample from a subject that has or is at risk for developing Gulf War Illness, wherein said substance is:
(i) one or more nucleic acid sequences selected from the group consisting of a miR-141 sequence, a miR-200b sequence, a miR-223 sequence, a miR-130a sequence, a miR-155 sequence, a miR-20a sequence, a miR-10a sequence, and any combination thereof; or
(ii) one or more nucleic acid amplification products selected from the group consisting of a miR-141 amplification product, a miR-200b amplification product, a miR-223 amplification product, a miR-130a amplification product, a miR-155 amplification product, a miR-20a amplification product, a miR-10a amplification product, or any combination thereof; and
(b) a detecting reagent or a detecting apparatus capable of detecting binding of said binding agent to said substance.

17. The kit of claim 16, wherein the binding agent is a probe capable of binding the nucleic acid sequence comprising miR-141, miR-200b, miR-223, miR-130a, miR-155, miR-20a, or miR-10a or the nucleic acid amplification product of miR-141, miR-200b, miR-223, miR-130a, miR-155, miR-20a, or miR-10a.

18. The kit of claim 17, wherein the probe is a labeled probe.

19. The kit of claim 16, comprising a detecting apparatus, wherein the detecting apparatus is an apparatus for performing a Northern blot analysis, an RT-PCR, a microarray analysis or a sequencing analysis.

20. The kit of claim 16, further comprising a control biological sample.


SEQUENCE LISTING (57 sequences)

SEQ ID NO: 1 - RNA, 22 nt, Homo sapiens
ugagguaguagguuguauaguu

SEQ ID NO: 2 - RNA, 22 nt, Homo sapiens
ugagguaguagguuguaugguu

SEQ ID NO: 3 - RNA, 22 nt, Homo sapiens
agagguaguagguugcauaguu

SEQ ID NO: 4 - RNA, 22 nt, Homo sapiens
cuauacgaccugcugccuuucu

SEQ ID NO: 5 - RNA, 22 nt, Homo sapiens
ugagguaggagguuguauaguu

SEQ ID NO: 6 - RNA, 23 nt, Homo sapiens
uacccuguagauccgaauuugug

SEQ ID NO: 7 - RNA, 22 nt, Homo sapiens
caaauucguaucuaggggaaua

SEQ ID NO: 8 - RNA, 23 nt, Homo sapiens
uacccuguagaaccgaauuugug

SEQ ID NO: 9 - RNA, 22 nt, Homo sapiens
acagauucgauucuaggggaau

SEQ ID NO: 10 - RNA, 22 nt, Homo sapiens
aagcccuuaccccaaaaagcau

SEQ ID NO: 11 - RNA, 21 nt, Homo sapiens
cuuuuugcggucugggcuugc

SEQ ID NO: 12 - RNA, 22 nt, Homo sapiens
cagugcaauguuaaaagggcau

SEQ ID NO: 13 - RNA, 23 nt, Homo sapiens
acuccauuuguuuugaugaugga

SEQ ID NO: 14 - RNA, 22 nt, Homo sapiens
uaacacugucugguaaagaugg

SEQ ID NO: 15 - RNA, 23 nt, Homo sapiens
uguaguguuuccuacuuuaugga

SEQ ID NO: 16 - RNA, 21 nt, Homo sapiens
cauaaaguagaaagcacuacu

SEQ ID NO: 17 - RNA, 23 nt, Homo sapiens
uuaaugcuaaucgugauaggggu

SEQ ID NO: 18 - RNA, 22 nt, Homo sapiens
cuccuacauauuagcauuaaca

SEQ ID NO: 19 - RNA, 23 nt, Homo sapiens
caaagugcuuacagugcagguag

SEQ ID NO: 20 - RNA, 24 nt, Homo sapiens
uuuggcaaugguagaacucacacu

SEQ ID NO: 21 - RNA, 21 nt, Homo sapiens
ugguucuagacuugccaacua

SEQ ID NO: 22 - RNA, 22 nt, Homo sapiens
uauggcacugguagaauucacu

SEQ ID NO: 23 - RNA, 22 nt, Homo sapiens
caaagaauucuccuuuugggcu

SEQ ID NO: 24 - RNA, 22 nt, Homo sapiens
gcccaaaggugaauuuuuuggg

SEQ ID NO: 25 - RNA, 22 nt, Homo sapiens
uaacacugucugguaacgaugu

SEQ ID NO: 26 - RNA, 22 nt, Homo sapiens
caucuuaccggacagugcugga

SEQ ID NO: 27 - RNA, 22 nt, Homo sapiens
uaauacugccugguaaugauga

SEQ ID NO: 28 - RNA, 23 nt, Homo sapiens
uaauacugccggguaaugaugga

SEQ ID NO: 29 - RNA, 22 nt, Homo sapiens
cgucuuacccagcaguguuugg

SEQ ID NO: 30 - RNA, 22 nt, Homo sapiens
uucccuuugucauccuaugccu

SEQ ID NO: 31 - RNA, 23 nt, Homo sapiens
uaaagugcuuauagugcagguag

SEQ ID NO: 32 - RNA, 22 nt, Homo sapiens
acugcauuaugagcacuuaaag

SEQ ID NO: 33 - RNA, 22 nt, Homo sapiens
aagcugccaguugaagaacugu

SEQ ID NO: 34 - RNA, 22 nt, Homo sapiens
ugucaguuugucaaauacccca

SEQ ID NO: 35 - RNA, 22 nt, Homo sapiens
cguguauuugacaagcugaguu

SEQ ID NO: 36 - RNA, 21 nt, Homo sapiens
uucacaguggcuaaguuccgc

SEQ ID NO: 37 - RNA, 22 nt, Homo sapiens
agggcuuagcugcuugugagca

SEQ ID NO: 38 - RNA, 21 nt, Homo sapiens
uucacaguggcuaaguucugc

SEQ ID NO: 39 - RNA, 22 nt, Homo sapiens
agagcuuagcugauuggugaac

SEQ ID NO: 40 - RNA, 22 nt, Homo sapiens
uguaaacauccucgacuggaag

SEQ ID NO: 41 - RNA, 22 nt, Homo sapiens
uguaaacauccccgacuggaag

SEQ ID NO: 42 - RNA, 22 nt, Homo sapiens
cuuucagucagauguuugcugc

SEQ ID NO: 43 - RNA, 22 nt, Homo sapiens
uguaaacauccuugacuggaag

SEQ ID NO: 44 - RNA, 23 nt, Homo sapiens
gaagugcuucgauuuuggggugu

SEQ ID NO: 45 - RNA, 22 nt, Homo sapiens
acucaaaaugggggcgcuuucc

SEQ ID NO: 46 - RNA, 22 nt, Homo sapiens
agaucagaaggugauuguggcu

SEQ ID NO: 47 - RNA, 23 nt, Homo sapiens
ucuuggaguaggucauugggugg

SEQ ID NO: 48 - RNA, 22 nt, Homo sapiens
aagacgggaggaaagaagggag

SEQ ID NO: 49 - RNA, 21 nt, Homo sapiens
cggggcagcucaguacaggau

SEQ ID NO: 50 - RNA, 22 nt, Homo sapiens
uccuguacugagcugccccgag

SEQ ID NO: 51 - RNA, 22 nt, Homo sapiens
uauugcacucgucccggccucc

SEQ ID NO: 52 - RNA, 22 nt, Homo sapiens
agggacgggacgcggugcagug

SEQ ID NO: 53 - RNA, 23 nt, Homo sapiens
caaagugcuguucgugcagguag

SEQ ID NO: 54 - RNA, 22 nt, Homo sapiens
acugcugagcuagcacuucccg

SEQ ID NO: 55 - RNA, 22 nt, Homo sapiens
cacccguagaaccgaccuugcg

SEQ ID NO: 56 - RNA, 24 nt, Homo sapiens
aggcaggggcuggugcugggcggg

SEQ ID NO: 57 - RNA, 22 nt, Homo sapiens
uucauucggcuguccagaugua